(19) United States
(12) Patent Application Publication
(10) Pub. No.: US 2026/0084286 A1
(43) Pub. Date: Mar. 26, 2026

(54) ROBOTIC JOINT INCLUDING A REMOTE CENTER OF MOTION MECHANISM

(71) Applicant: Bioness Medical, Inc., Valencia, CA (US)

(72) Inventors: Michael M. HOGAN, Austin, TX (US); William WU, Austin, TX (US); Rohit John VARGHESE, Austin, TX (US)

(21) Appl. No.: 19/123,917

(22) PCT Filed: Oct. 25, 2023
(86) PCT No.: PCT/US2023/077716

§ 371 Date: Apr. 24, 2025

Related U.S. Application Data
(60) Provisional application No. 63/419,377, filed on Oct. 26, 2022.

(51) Int. Cl. B25J 9/00; B25J 9/10; B25J 13/08

(52) U.S. Cl. CPC B25J 9/0006; B25J 9/1065; B25J 13/085

(57) ABSTRACT

An embodiment includes an exoskeleton robotic system comprising a parallelogram-based remote center of motion (RCM) joint. The RCM mechanism includes a first link that is coupled to a second link, a third link, and a fourth link. A motor is coupled to the third link. A first of the first, second, third, or fourth links includes a first flexure. The first flexure includes a first degree of freedom in a first direction. A second of the first, second, third, or fourth links includes a second flexure, the second flexure including a second degree of freedom in a second direction. The first direction is not parallel to the second direction.

### CROSS-REFERENCE TO RELATED APPLICATION

This application claims priority to United States Provisional Ser. No. 63/419,377 filed on Oct. 26, 2022 and entitled “Robotic Joint Including a Remote Center of Motion Mechanism”, the content of which is hereby incorporated by reference.

### TECHNICAL FIELD

Embodiments of the invention are in the field of robotics and, in particular, remote center of motion (RCM) mechanisms.

### BACKGROUND

As addressed in U.S. Pat. No. 10,463,560, exoskeletons are mechatronic systems worn by a person in such a way that a direct transfer of mechanical power from the exoskeleton occurs. These robotic mechanisms have been applied in a variety of settings, for example, telemanipulation, man-amplification, rehabilitation, and to assist impaired human motor control. However, many of these applications of exoskeleton devices have yet to find widespread use, acceptance, or practicality.

One example area in which these devices have been proposed is the treatment of stroke. Stroke affects thousands of Americans every year and the recovery process is long, difficult, and costly. The use of a robotic exoskeleton may potentially reduce the length, difficulty, and cost of this recovery process. Various efforts have been proposed to provide a robotic exoskeleton for the upper-body.

Such exoskeletons may use parallelogram based remote center of motion (RCM) mechanisms, which generate a desired rotation about a center point where there exists no physical joint.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1A is a perspective view of an embodiment. FIGS. 1B, 1C, and 1D are top views of an embodiment.

FIGS. 2A, 2B are perspective views of an embodiment.

FIG. 3A is a top view of an embodiment. FIG. 3B is a perspective view of an embodiment.

FIG. 4A is a top view of an embodiment. FIG. 4B is a perspective view of an embodiment.

FIGS. 5A, 5B, 5C, 5D are top views of embodiments.

FIG. 6A is a perspective view of an embodiment. FIG. 6B is a side view of an embodiment.

FIGS. 7A and 7B are top views of embodiments.

FIGS. 8A, 8B, 8C, and 8D are models of a parallelogram RCM.

FIG. 9A is a perspective view of an embodiment. FIGS. 9B, 9C are bottom and top views of the embodiment. FIGS. 9D, 9E, 9F are perspective views of the embodiment. FIG. 9G is a side view of an embodiment.

FIGS. 10A, 10B, 10C are bottom views of an RCM mechanism.

FIGS. 11A and 11B illustrate structures that disagree and agree with the Mobility Equation.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical contact with each other and “coupled” may indicate elements co-operate or interact with each other, but they may or may not be in direct physical contact. Phrases such as “comprising at least one of A or B” include situations with A, B, or A and B.

Applicant determined physical therapy for rehabilitation of neurological and neuromuscular disorders has many shortcomings in its current manual techniques. Robotic therapy devices have potential to provide beneficial improvements, but are limited by their requirements for systems that require extremely precise and expensive manufacturing techniques to ensure proper operation of the systems. Embodiments described herein address these problems.

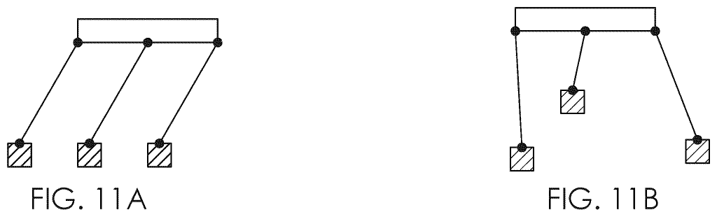

A series of definitions are now listed. A “remote center of motion” (RCM) is an axis of rotation remote from any bearings or mechanical supports. A “degree of freedom” (DOF) is the number of independent parameters (measurements) needed to uniquely define a system's position in space at any instant in time. A “binary link” is a linkage comprising two joints and a “ternary link” is a linkage comprising three joints. The “Mobility Equation for Planar Mechanisms” is M=3L−2J−3G (where M=predicted degrees of freedom, L=number of links including grounded links, J=number of joints, and G=number of grounded links). An “overconstrained mechanism” is a mechanism with more degrees of freedom than predicted by the mobility formula. The overconstraint occurs due to the presence of redundant joints in the mechanism. See, e.g., FIG. 11B (where DOF=0 agrees with the Mobility Equation) and FIG. 11A (where DOF=1 disagrees with the Mobility Equation due to the unique geometry of the device). A “redundant joint” is a joint that constrains the same degrees of freedom as another joint(s) in the same mechanism. A “revolute joint” is a 1-DOF joint that constrains two links to rotation about an axis. A “prismatic joint” is a 1-DOF joint that constrains two links to translation along an axis. A “change point” occurs when members of a parallelogram become aligned, allowing for the parallelogram mechanism to change form from a parallelogram to an antiparallelogram form. A “flexure” is a resilient member configured to flex and accommodate stress and/or strain from misaligned or misoriented features.

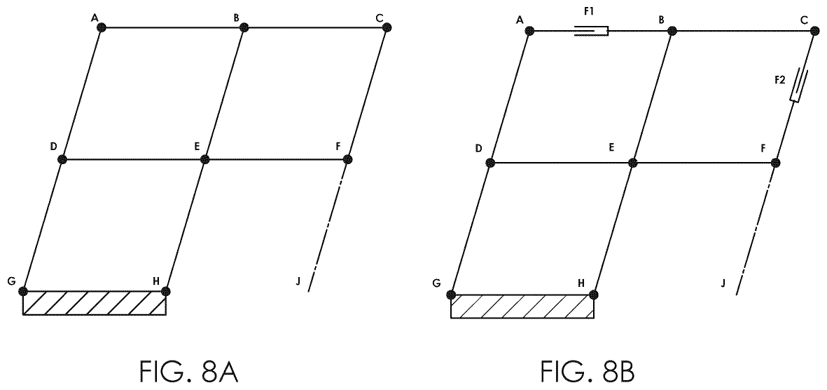

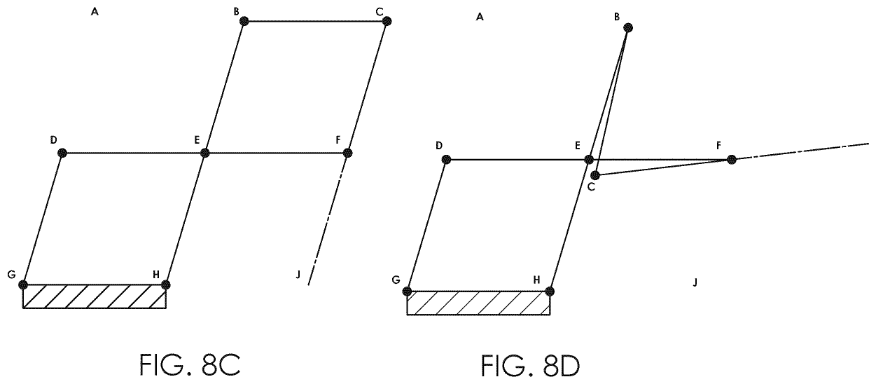

The mobility equation for planar mechanisms is sufficient to determine whether a planar system is an “overconstrained mechanism”. For example, FIG. 8A depicts a model of a planar parallelogram-based RCM mechanism. The RCM mechanism includes joints (A, B, C, D, E, F, G, H), binary links (G-H, C-F), ternary links (A-C, D-F, A-G, B-H), and RCM (J). Link G-H is the grounded link. The RCM mechanism includes redundant joints A, B, D, E resulting from the parallelogram arrangement of the mechanism.

The RCM mechanism of FIG. 8A is overconstrained because of the redundant joints, which cause the RCM mechanism to have 1 DOF about the RCM J despite having −1 predicted DOF from the planar mobility equation. FIGS. 8C and 8D show a similar planar parallelogram-based RCM mechanism without joint A. Removing a single joint has the effect of increasing the predicted DOF from the planar mobility equation by 2. As such, the system has a predicted DOF of 1 and is therefore not overconstrained. A single mechanism of this type can operate in both parallel (FIG. 8C) and antiparallel (FIG. 8D) forms. When links B-H and C-F become colinear the mechanism is operating at a change point, and the system may freely switch between the parallel and antiparallel forms. This unpredictable behavior is undesirable in the embodied design because link C-F no longer rotates about J in the antiparallel form. The presence of the redundant joint A enforces the parallelism of links A-C and D-F to prevent the mechanism from entering the antiparallel form at its change points. FIG. 8B depicts a parallelogram-based RCM mechanism with additional prismatic joints F1 and F2. Link A-B-C becomes pseudo-links A-F1 and F1-B1-C while link A-D-G becomes pseudo-links A-F2and F2-D1-C. These are referred to as pseudo-links because the joints F1 and F2 only permit small displacements. This mechanism has two more pseudo-links and two more joints than the mechanism depicted in FIG. 8A, and therefore has two additional predicted degrees of freedom without the antiparallel form associated with FIG. 8D. For clarity, the terms “ternary link” and “binary link” will be used to refer to linkages as if there were no flexure mechanisms present, since such terms still describe the dominant behavior of the mechanism. For example, in FIG. 8B link A-B-C is still referred to a “ternary link” despite the A-F1 binary link and F1-B-C ternary link.

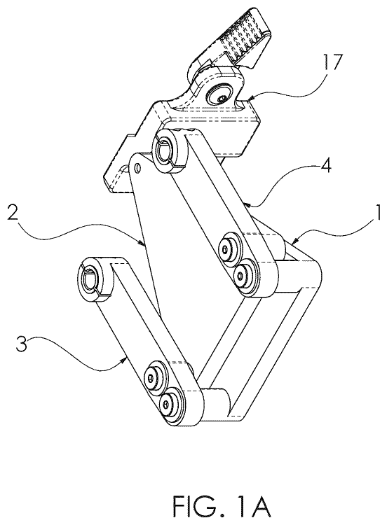

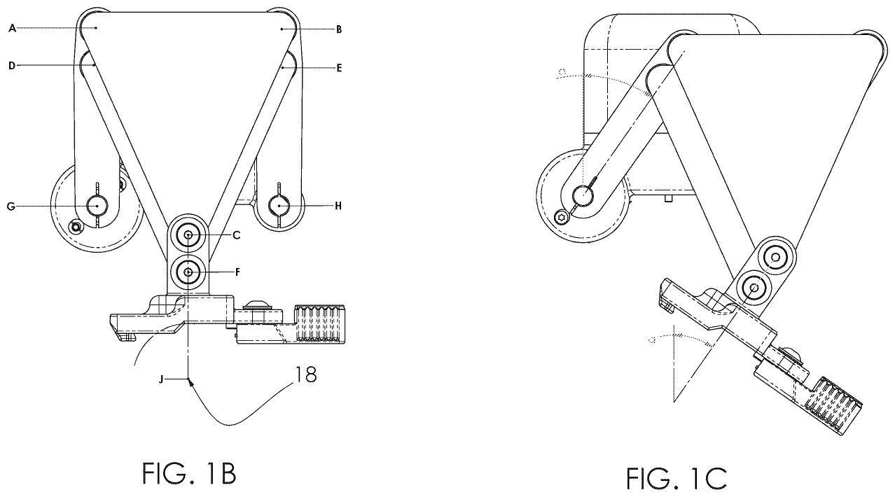

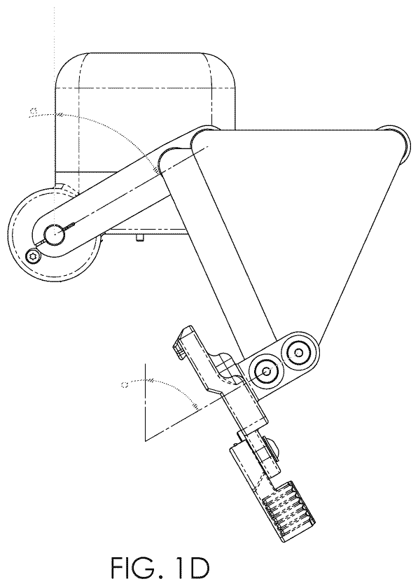

FIGS. 1A, 1B, 1C depict a physical embodiment of a parallelogram-based RCM mechanism coupled to bracket 17. The mechanism includes links 1, 2, 3, 4 and a remote axis of rotation 18. The RCM mechanism is analogous to the mechanism depicted in FIG. 8A and includes joints (A, B, C, D, E, F, G, H), binary links (G-H, C-F), ternary links (A-B-C, D-E-F, A-D-G, B-E-H), and RCM (J). Applicant determined such an embodiment is appropriate for some applications. However, Applicant further determined that due to the nature of the parallelogram structure, there exist redundant joints which result in an overconstrained mechanism. Such redundant joints include joints A, B, D, E.

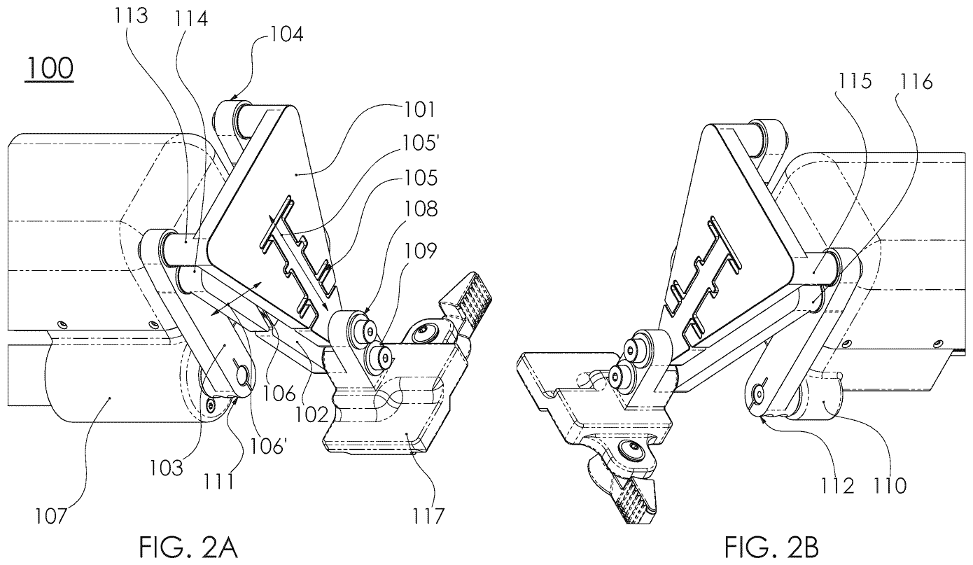

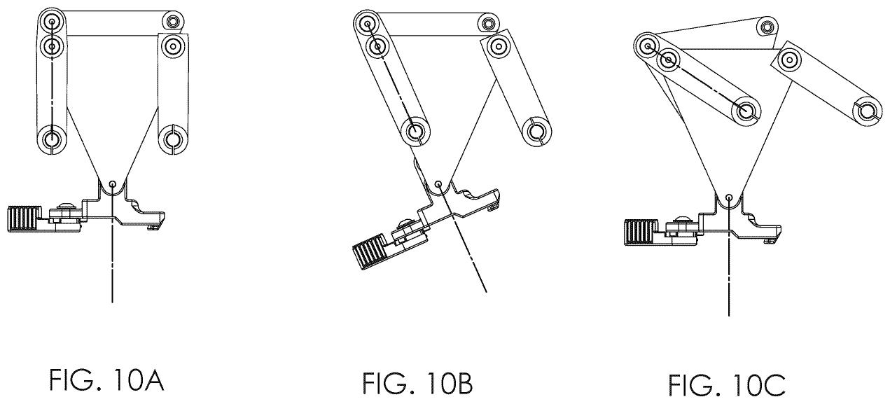

As shown in FIGS. 1B, and 1C, each set of links remains parallel during movement of an overconstrained parallelogram RCM mechanism. However, inaccuracy caused by the imperfect manufacture of the mechanism may cause the linkages to shift slightly out of parallel, violating the unique parallelogram arrangement of joints needed such that they could be considered redundant. For rigid links, this shifting may cause the mechanism to bind or else impart additional forces which may interfere with any torque sensing (e.g., see sensor 110 of FIG. 2B) which may be integrated with the RCM mechanism. See also FIG. 10A, which depicts another physical embodiment that is analogous to FIG. 8C, where joint A has been removed. Such an embodiment is subject to entering an antiparallel form as depicted in FIGS. 10C and 8D. A change point arises when one ternary link (e.g., B-E-H) overlaps with a binary link (e.g., C-F). This antiparallel form (FIG. 8D) deviates from the desired rotation about the RCM J which prevents usability as a therapeutic device.

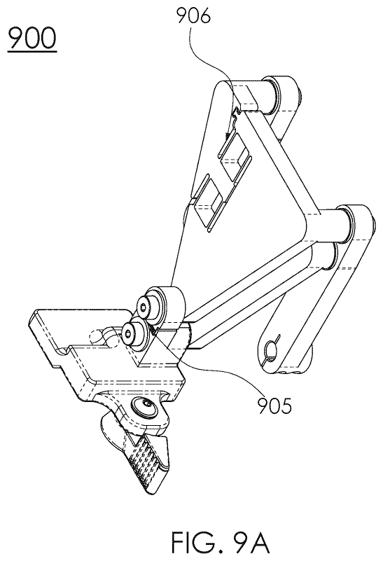

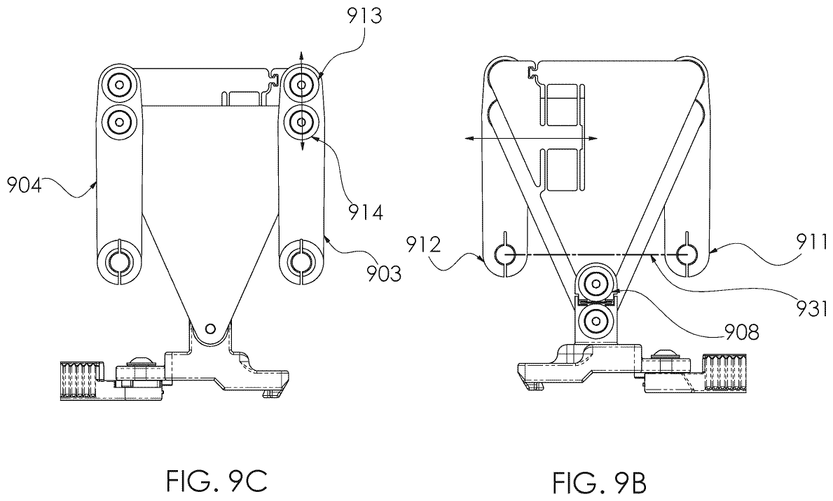

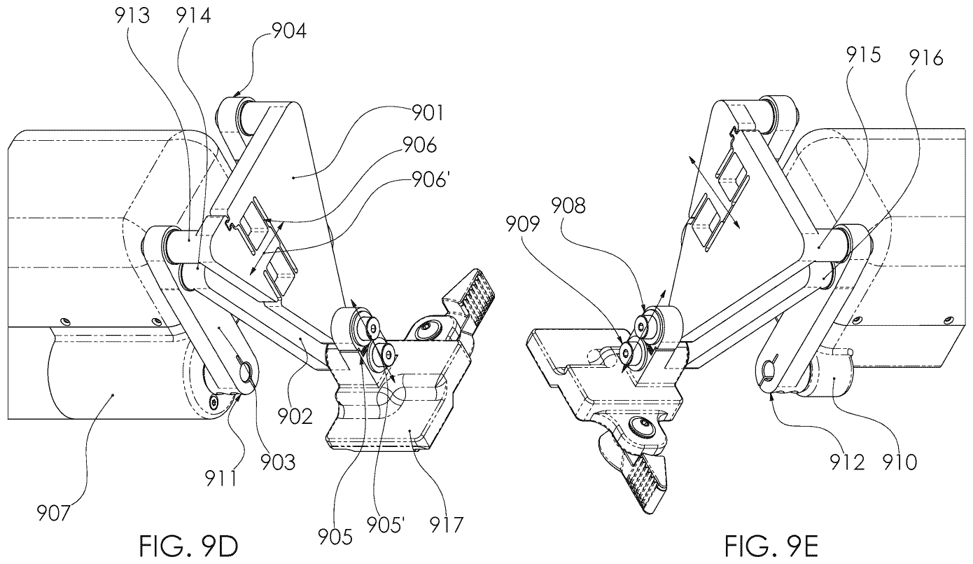

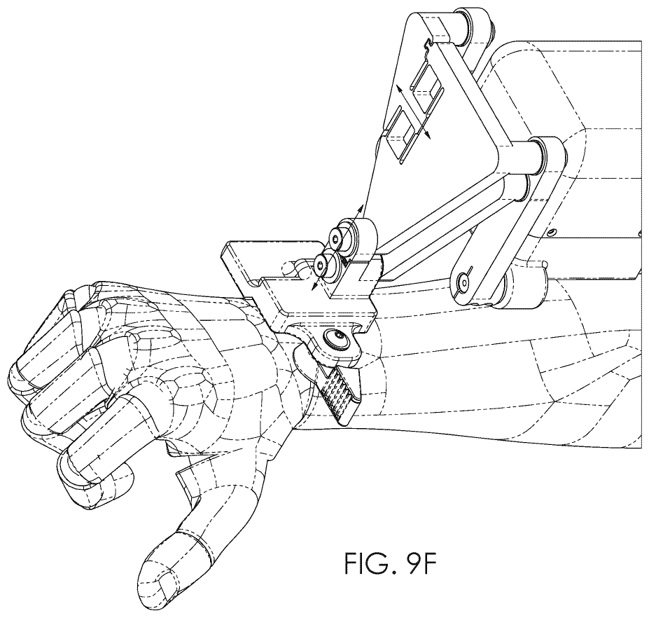

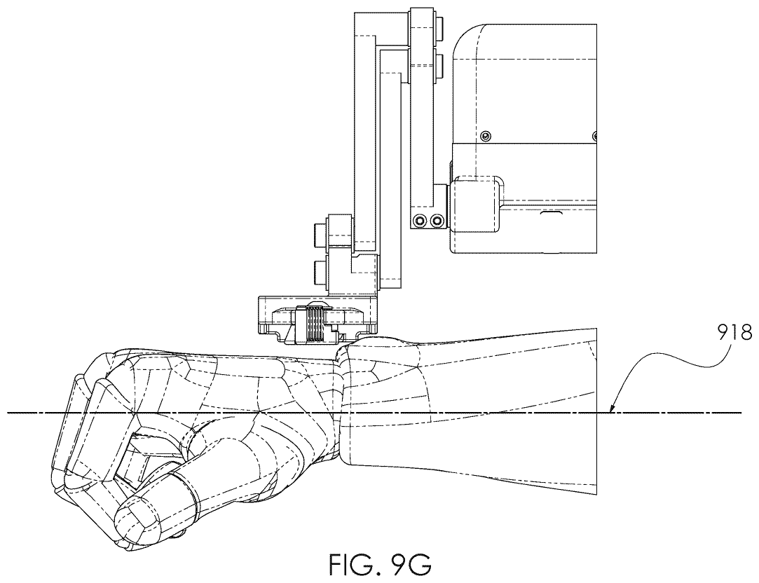

However, FIG. 9A includes an embodiment that addresses shortcomings of the systems of FIGS. 1A, 10A. Namely, FIG. 9A includes flexures 905, 906. Each flexure constitutes the addition of a prismatic joint to ternary link A-B-C and binary link C-F and as a result has two additional predicted degrees of freedom. Because each flexure only permits motion along a single direction, the parallelogram-based RCM mechanism is able to manage overconstraint in the system without allowing the mechanism to operate in the antiparallelogram form. Both 1-DOF flexure mechanisms can be integrated in any of the binary or ternary links but are arranged such that their directions of motion do not become parallel throughout the intended range of motion of the mechanism.

In contrast, FIGS. 10A, 10B, 10C show a system wherein a ternary link of FIG. 9A is replaced with a binary link, thereby removing a redundant joint. Notice how in FIG. 10C the lack of redundant joint causes a failure at a change point and the RCM mechanism enters an antiparallel state (i.e., the parallelogram RCM mechanism takes an antiparallelogram form). This antiparallel state is avoided with the embodiment of, for example, FIG. 9A. As a result, the RCM mechanism maintains its predictable rotation about a remote axis of rotation. The predictability is critical in certain applications, such as robotic-based physical rehabilitation. FIGS. 1A through 1D show a mechanism travelling through change points and not binding or entering an antiparallel state. However, such a system must have extremely exact tolerances to operation in such a fashion. Thus, the embodiments of, for example, FIGS. 2A and 9A with their flexures both avoid entering an antiparallel state at change points while also allowing for possible manufacturing errors/less exact manufacturing tolerances.

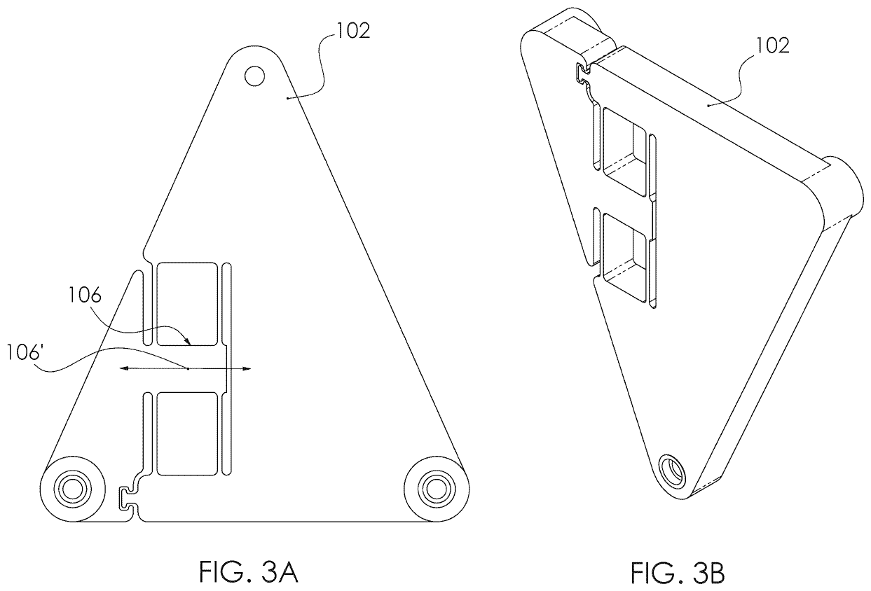

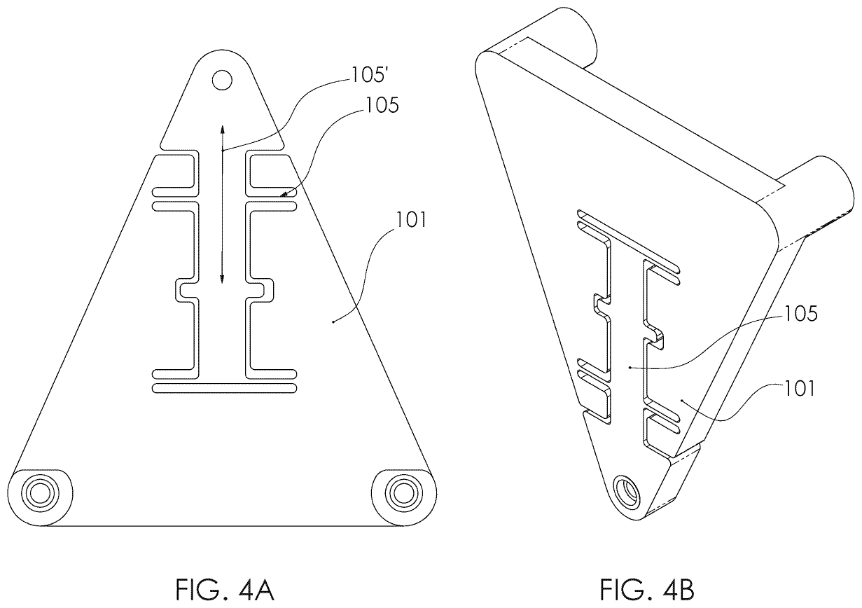

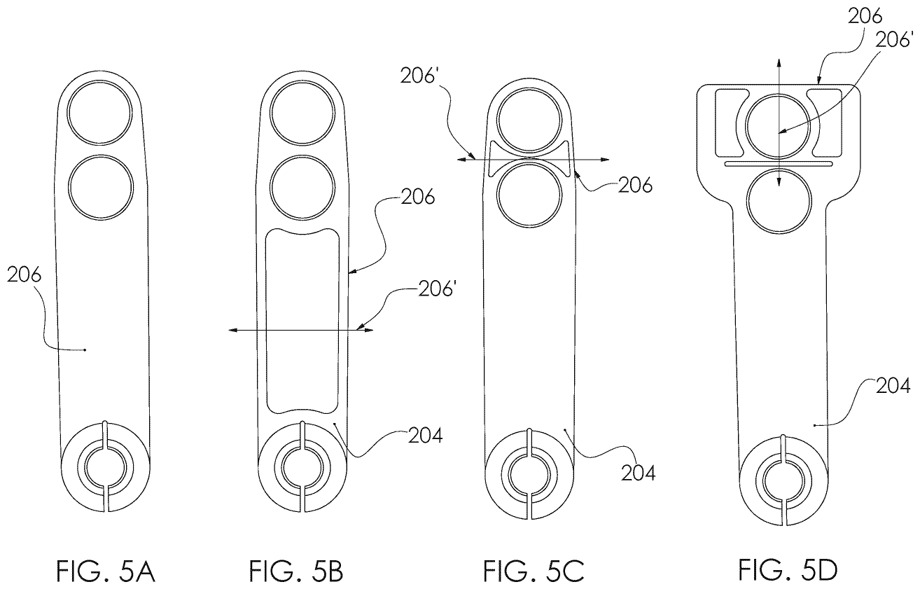

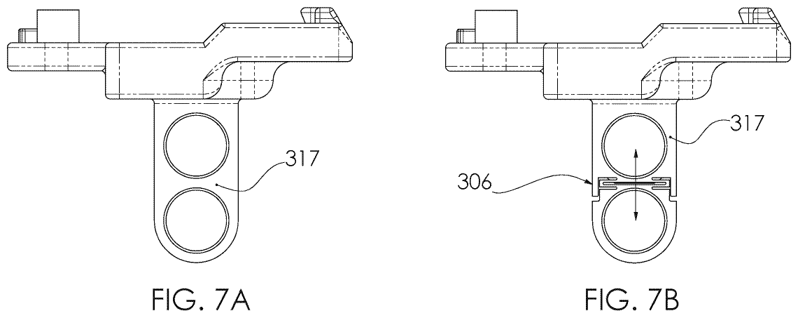

With integration of flexures 905, 906 of FIG. 9A, the movement of the redundant joints away from their perfect locations relative to the other redundant joints (possibly due to manufacturing error or tolerances) is allowed for by the deflection of the flexures. These flexures can be implemented in any of the binary or ternary links. See, for example, the ternary links of FIGS. 5C, 5D. The flexure depicted in FIG. 3A can be integrated in either ternary link A-B-C or D-E-F (which are labeled on an analogous device in FIG. 1B). Flexures like that depicted in FIG. 7B can be implemented on either binary link C-F or G-H. FIG. 7A shows a binary link without any flexure mechanism. FIG. 5A shows a ternary link without any flexure mechanism built in. Thus, the links that include flexure mechanisms may vary with different embodiments.

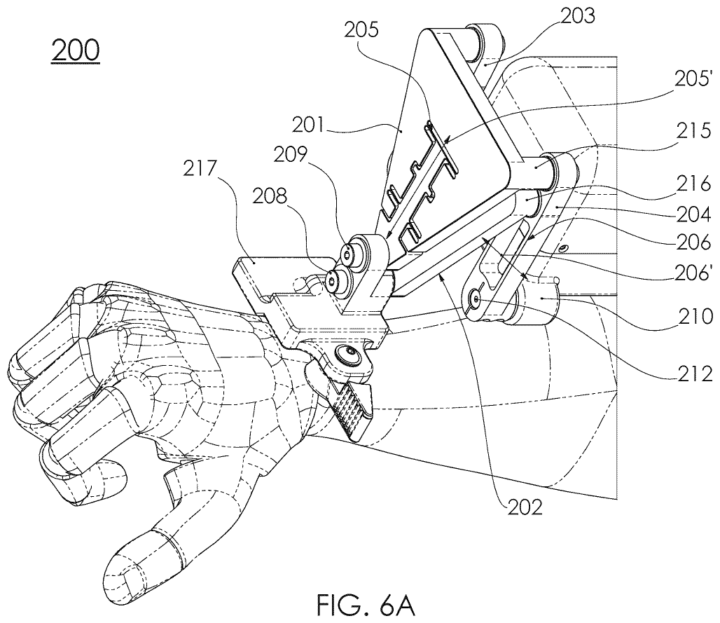

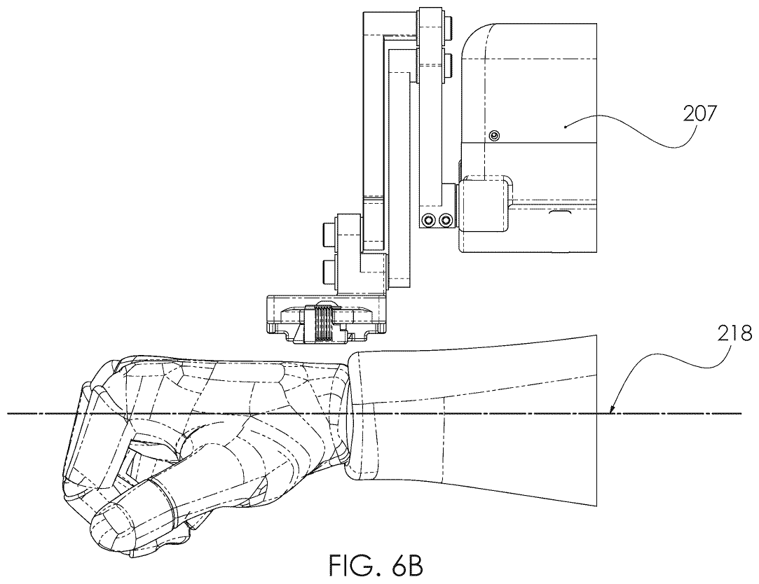

However, the selection of any two single degree of freedom flexure mechanisms helps manage the over constraint provided in an RCM mechanism provided that the direction of action of the flexure mechanisms are not parallel. For instance, in FIG. 9B fixed revolute joints 911, 912 may be separated from each other by distance 931. Distance 931 may deviate from the desired manufacturing tolerances. Flexure 906 may limit the ramifications of the manufacturing error by allowing flexibility 906′ for joint 914. In FIG. 9D one or both of members 903, 904 may be longer or shorter than called for in desired manufacturing tolerances. Flexure 905 may limit the ramifications of the manufacturing error by allowing vertical flexibility 905′ for joint 908. Further examples are shown in FIGS. 2A, 2B, and 6A.

With flexure-based embodiments described herein, deviation from perfectly located joints results in negligible imperfections to the overall RCM motion in applications like physical therapy (where the required precision of RCM motion is low). Additionally, embodiments improve the force sensitivity of RCM mechanisms by significantly reducing any forces which would have resulted from overconstraint within the mechanism. This increase in force sensitivity is critical to applications such as physical therapy robotics.

Regarding various applications of embodiments, the motion of RCMs described herein are useful for medical device applications where motions need to be centered about anatomical joints or locations. These may include rehabilitation exoskeletons, robotic surgery systems, and the like. However, applications may extend to general robotics and the like. Further, whereas embodiments described herein are described with respect to the joints of the upper limb, it will be appreciated that the embodiments may be configured for use with other joints, such as, for example, the hip or knee and may have N degrees of freedom (i.e., single DOF, two DOF, three DOF).

An example of where these mechanisms are utilized are in the wrist mechanism of rehabilitation robotics. In such an application, a parallelogram RCM mechanism generates one rotational degree of freedom aligned with the anatomical forearm pronation/supination axis. A mechanism, such as the mechanism of FIG. 1A, utilizes two parallelograms which share several pivots. The mechanism is overconstrained. As a result, loads and stresses on the RCM mechanism are partially dependent on the manufacturing accuracy of the RCM mechanism. For example, if links 3, 4 of FIG. 1A are not the same length, in precision force sensing applications this would be unacceptable because the over constraint imposes additional forces, torques, and friction on the system which may interfere with the system's force sensing ability.

However, flexures 105, 106 of FIG. 2A manage this overconstraint problem by introducing two additional degrees of freedom by the addition of two separate one DOF flexure mechanisms that allow the redundant pivots (i.e., revolute joints) to displace independently. By allowing for the displacement of the redundant pivots/joints in the parallelogram RCM mechanism, manufacturing errors in the linkages result in displacements of the pivots/joints instead of forces, torques, or friction. The displacements of the pivots/joints have no additional detrimental effects on the RCM mechanism.

The following examples pertain to further embodiments.

Example 1 includes an exoskeleton robotic system comprising several components including a parallelogram-based remote center of motion (RCM) mechanism (100). The RCM mechanism includes a first ternary link (101) that is coupled to a second ternary link (102), a third ternary link (103), a fourth ternary link (104), a first binary link (117), and a second binary link. Motor (107) is coupled to the third ternary link and sensor (110) is coupled to the fourth ternary link. The first ternary link includes a first flexure (105), the first flexure configured to act in a first direction (105′). The second ternary link includes a second flexure (106), the second flexure configured to act in a second direction (106′). The first direction is not parallel to the second direction.

See, for example, FIGS. 2A-2B. For example, the sensor may include a position sensor, torque sensor, or combinations thereof. In an embodiment, sensor 110 is a position sensor and a torque sensor is included within the motor on joint 111.

Sensor 110 may include a plurality of sensors or at least one link that combine or collectively sense elements such as position, torque, and the like. In an embodiment, any of the links may include a plurality of links or at least one link.

Example 2. The exoskeleton robotic system of example 1, wherein in response to actuation by the motor the RCM mechanism is configured to rotate about a remote center axis located within a body of a user of the exoskeleton robotic system.

Example 3. The exoskeleton robotic system of example 2, wherein the RCM mechanism includes only one degree of freedom. The only one degree of freedom is a rotational degree of freedom about the remote center axis. The RCM mechanism does not include a second degree of freedom.

For example, the embodiment of FIG. 11A has 1 DOF. Adding flexures, such as flexures 105 or 106, is the same as adding one joint and one link for each flexure. Doing so brings the predicted DOF into agreement with the actual single DOF about the RCM axis. In FIG. 11A, the RCM mechanism's one degree of freedom is a rotational degree of freedom. However, degrees of freedom such as translational degrees of freedom or the remaining two rotational degrees of freedom are not present. However, due to flexures 105 and 106, for the embodiment of FIG. 2A the predicted DOF is M=3(6+2)−2(8+2)−3(1)=1. The number of links is increased by 2 due to flexures 105, 106 and the number of joints is increased by 2 due to flexures 105, 106.

Example 4. The exoskeleton robotic system according to any of examples 1-3, wherein the RCM mechanism is an RCM mechanism that includes at least two redundant joints.

Another version of example 4. The exoskeleton robotic system according to any of examples 1-3, wherein the RCM mechanism is a parallelogram-based RCM mechanism that includes at least two redundant joints. The at least two redundant joints are located on at least two of the first, second, third, and fourth ternary links.

Example 5. The exoskeleton robotic system according to any of examples 1-4, wherein the RCM mechanism is not over constrained.

Because the mechanism is not over constrained, manufacturing tolerances are lessened thereby promoting easier and more cost-efficient manufacturing.

For example, the mobility equation for the RCM mechanism of FIG. 2A (but without flexures 105, 106) would be M=3(6)−2(8)−3(1)=−1. However, with the flexures M=1. This agrees with the actual DOF considering the RCM mechanism clearly rotates about the RCM (so the RCM mechanism has 1 DOF). By definition, this agreement between predicted DOF (based on the mobility equation) and actual DOF defines the RCM mechanism as not overconstrained. Thus, embodiments such as those of FIGS. 2A, 6A, 9A are not overconstrained. These embodiments include redundant joints, which prevent the antiparallelogram form. Flexures such as flexures 105, 106 “free up” the system to avoid binding. This is evident by the fact that the mobility equation now equals 1 so the system is not overconstrained by definition. The arrangement of flexures in embodiments such as FIG. 9A is such that the flexure's direction of flexure do not overlap throughout the range of rotation of the mechanism about the RCM. For example, the embodiment of FIG. 9A may accommodate a 144° range of motion. Alternative embodiments include any arrangement of two flexures on binary and/or ternary links, provided that their directions do not become parallel throughout the intended range of motion of the mechanism about the RCM.

While many embodiments address systems that are not over constrained, other embodiments may be overconstrained. For example, see FIG. 1A. By allowing for embodiments that are constrained or not constrained, the designer has a choice regarding how to best manage manufacturing options.

Example 6. The exoskeleton robotic system according to any of examples 1-5, wherein the first binary link includes a bracket. The bracket couples to the first ternary link via a first revolute joint (108) and the bracket couples to the second ternary link via a second revolute joint (109).

The bracket may be one of several brackets.

Example 7. The exoskeleton robotic system according to any of examples 1-5, wherein the first binary link includes a bracket. The bracket couples to the first ternary link via a first revolute joint (108) and the bracket couples to the second ternary link via a second revolute joint (109). The third ternary link couples to the first ternary link via a third revolute joint (113). The third ternary link couples to the second ternary link via a fourth revolute joint (114). The fourth ternary link couples to the first ternary link via a fifth revolute joint (115). The fourth ternary link couples to the second ternary link via a sixth revolute joint (116).

In an embodiment, each of the six links (101, 102, 103, 104, 117, and 118) may be rigid.

Example 8. The exoskeleton robotic system according to any of examples 1-7, wherein the first direction is orthogonal to the second direction.

Another version of example 8. The exoskeleton robotic system according to any of examples 1-7, wherein the first direction is not parallel to the second direction throughout the RCM mechanism's range of motion.

Example 9. The exoskeleton robotic system according to any of examples 1-8, wherein the sensor includes a torque sensor. The motor and the torque sensor couple to the RCM mechanism via different revolute joints.

The torque sensor may be one of several torque sensors.

Another version of example 9. The exoskeleton robotic system according to any of examples 1-8, comprising an additional sensor. The additional sensor includes a torque sensor and the motor and the torque sensor couple to the RCM mechanism via a single revolute joint.

For example, in an embodiment the motor and a torque sensor couple to the RCM mechanism via the same revolute joint. In the same embodiment a position sensor (110) and the motor couple to the RCM mechanism via different revolute joints. An embodiment may include both torque and position sensors.

In an embodiment the motor and a first sensor couple to the RCM mechanism via the same revolute joint and the motor and a second sensor couple to the RCM mechanism via different revolute joints. In an embodiment the first sensor is a torque sensor and the second sensor is a position sensor. In an embodiment the second sensor is a torque sensor and the first sensor is a position sensor.

For example, see revolute joints 111, 112. Sensors (e.g., encoders, torque sensors, accelerometers) may be used to obtain data relating to position, angle, acceleration, force (e.g., torque), etc., of one or more joints and/or segments the limb may be deployed on and/or associated with the mechanical linkage of the exoskeleton.

Embodiments improve the performance of the torque-sensing ability of the system since binding forces are undesirable and taint force measurements that target the patient (and embodiments lessen binding).

Example 10. The exoskeleton robotic system according to any of examples 1-9, wherein the RCM mechanism is a robotic shoulder joint or a robotic wrist joint.

Example 11. The exoskeleton robotic system of example 1, wherein the RCM mechanism is a parallelogram-based RCM mechanism. In response to actuation by the motor the RCM mechanism is configured to rotate about a center axis located within a body of a user of the exoskeleton robotic system. The RCM mechanism is configured to rotate within a rotation range that is at least 40 degrees and less than 180 degrees.

The rotation range can change depending on the length of the links. Other embodiments are not limited to this degree range.

Another version of example 11. The exoskeleton robotic system of example 1, wherein the RCM mechanism is a parallelogram-based RCM mechanism. In response to actuation by the motor the RCM mechanism is configured to rotate about a center axis located within a body of a user of the exoskeleton robotic system. The RCM mechanism is configured to rotate through an entirety of a rotation range that includes a change point.

Example 11.1 The exoskeleton robotic system according to example 11, wherein the first direction is never parallel to the second direction throughout the entirety of the rotation range.

Example 12. The exoskeleton robotic system according to any of examples 1-11, wherein: the first ternary link is primarily disposed in a first plane; the second ternary link is primarily disposed in a second plane; the first plane is parallel to the second plane.

Example 1a. An exoskeleton robotic system comprising a parallelogram-based remote center of motion (RCM) joint (200). The RCM mechanism includes a first link (201) that is coupled to a second link (202), a third link (203), and a fourth link (204). A motor (207) is coupled to the third link. A first of the first, second, third, or fourth links includes a first flexure (205), the first flexure (205) including a first degree of freedom in a first direction (205′). A second of the first, second, third, or fourth links includes a second flexure (206), the second flexure including a second degree of freedom in a second direction (206′). The first direction is not parallel to the second direction.

See, for example, FIG. 6A. Thus, embodiments vary and may include an RCM mechanism comprised of any two flexures on any two links not acting parallel.

Example 2a. The exoskeleton robotic system of example 1a, wherein the first link is a first ternary link and the second link is a second ternary link.

Example 2.1a. The exoskeleton robotic system according to example 2a, wherein: the first flexure is included in one of the first or second links; and the second flexure is included in one of the third or fourth links.

For example, the third or fourth link may include any of the links of FIGS. 5B, 5C, or 5D. See also, FIGS. 6A, 6B. Thus, while the embodiment of FIG. 2A includes flexures in the ternary links other embodiments may include flexures in other links. While FIG. 6B does not show the hand/wrist directly connected to the joint, a connector (not shown) may couple the patient's hand/wrist to the joint.

For example, the third or fourth link may include any of the links of FIG. 9A, such as the binary link including flexure 905.

Example 2.2a. The exoskeleton robotic system according to any of examples 1a-2.1a, wherein in response to actuation by the motor the RCM mechanism is configured to rotate about a center point located within a body of a user of the exoskeleton robotic system.

For example, the center point may be located on axis 218.

Example 3a. The exoskeleton robotic system of example 2a, wherein: the RCM mechanism includes only one degree of freedom. The only one degree of freedom is a rotational degree of freedom about the center point. The RCM mechanism does not include a second degree of freedom.

Example 4a. The exoskeleton robotic system according to any of examples 1a-3a, wherein the RCM mechanism includes at least two redundant joints.

Another version of example 4a. The exoskeleton robotic system according to any of examples 1a-3a, wherein the RCM mechanism includes at least two redundant joints. The at least two redundant joints are collectively located on the first and second links.

The flexure mechanisms can be integrated into any of the binary or ternary links but are arranged such that their directions of motion do not become parallel throughout the intended range of motion of the mechanism.

Example 5a. The exoskeleton robotic system according to any of examples 1a-4a, wherein the RCM mechanism is not over constrained.

Example 6a. The exoskeleton robotic system according to any of examples 1a-5a, comprising a fifth link. The fifth link includes a bracket. The bracket couples to the first link via a first revolute joint (208). The bracket couples to the second link via a second revolute joint (209).

In an embodiment, a bracket such as bracket 317 of FIG. 7B may include flexure 306 of FIG. 7B. This may remove the need for a flexure such as flexure 205 of FIG. 6A if the flexures would each have a degree of freedom parallel to each other. FIG. 7A includes a bracket with no flexure.

Example 7a. The exoskeleton robotic system according to any of examples 1a-5a comprising a fifth link. The fifth link includes a bracket that couples to the first link via a first revolute joint (208). The bracket couples to the second link via a second revolute joint (209). The third link couples to the first link via a third revolute joint. The third link couples to the second link via a fourth revolute joint. The fourth link couples to the first link via a fifth revolute joint (215). The fourth link couples to the second link via a sixth revolute joint (216).

Example 8a. The exoskeleton robotic system according to any of examples 1a-7a, wherein in one orientation the first direction is orthogonal to the second direction.

Another version of example 8a. The exoskeleton robotic system according to any of examples 1a-7a, wherein the first direction is not parallel to the second direction throughout the RCM mechanism's range of motion.

Example 9a. The exoskeleton robotic system according to any of examples 1a-8a comprising a sensor (210), wherein the motor and the sensor couple to the RCM mechanism via different revolute joints.

For example, see revolute joint 212.

Another version of example 9a. The exoskeleton robotic system according to any of examples 1a-8a comprising a sensor, wherein the motor and the sensor couple to the RCM mechanism via a single revolute joint.

Example 10a. The exoskeleton robotic system according to any of examples 1a-9a, wherein the RCM mechanism is a robotic shoulder joint or a robotic wrist joint.

Example 11a. The exoskeleton robotic system of example 1a, wherein in response to actuation by the motor the RCM mechanism is configured to rotate about a center axis located within a body of a user of the exoskeleton robotic system. The RCM mechanism is configured to rotate within a rotation range that is at least 40 degrees and less than 360 degrees.

Another version of example 11a. The exoskeleton robotic system of example 1a, wherein: the RCM mechanism is a parallelogram-based RCM mechanism. In response to actuation by the motor the RCM mechanism is configured to rotate about a center axis located within a body of a user of the exoskeleton robotic system. The RCM mechanism is configured to rotate through an entirety of a rotation range that includes a change point.

Example 11.1a The exoskeleton robotic system according to example 11a, wherein the first direction is never parallel to the second direction throughout the entirety of the rotation range.

Example 12a. The exoskeleton robotic system according to any of examples 1a-11a, wherein: the first link is a ternary link primarily disposed in a first plane; the second link is a ternary link primarily disposed in a second plane; the first plane is parallel to the second plane.

Example 1b. An exoskeleton robotic system comprising a remote center of motion (RCM) robotic joint (100). The RCM mechanism includes a first link (101) that is coupled to a second link (102), a third link (103), and a fourth link (104). A motor (107) is coupled to the third link. The first link includes a first plate with three pivot joints and the second link includes a second plate with three pivot joints. The first link includes a first resilient member (105) that is configured to flex in a first direction (105′) but not a second direction (106′). The second link includes a second resilient member (106) that is configured to flex in the second direction but not the first direction. The first direction is not parallel to the second direction.

Example 2b. The exoskeleton robotic system of example 1b, wherein in response to actuation by the motor the RCM mechanism is configured to rotate about a center point located within a body of a user of the exoskeleton robotic system.

Example 3b. The exoskeleton robotic system of example 2b, wherein: the RCM mechanism includes only one degree of freedom. The only one degree of freedom is a rotational degree of freedom about the center point. The RCM mechanism does not include a second degree of freedom.

Example 4b. The exoskeleton robotic system according to any of examples 1b-3b, wherein the RCM mechanism includes at least two redundant joints.

Another version of example 4. The exoskeleton robotic system according to any of examples 1-3, wherein the RCM mechanism includes at least two redundant joints, the at least two redundant joints being included on the first and second links.

Example 5b. The exoskeleton robotic system according to any of examples 1b-4b, wherein the RCM mechanism is not over constrained.

Example 6b. The exoskeleton robotic system according to any of examples 1b-5b comprising a bracket (117), wherein: the bracket couples to the first link via a first of the first link's three pivot joints (108). The bracket couples to the second link via a first of the second link's three pivot joints (109).

Example 7b. The exoskeleton robotic system according to any of examples 1b-5b comprising a bracket (117). The bracket couples to the first ternary link via a first of the first link's three pivot joints (108). The bracket couples to the second ternary link via a first of the second link's three pivot joints (109). The third link couples to the first link via a second of the first link's three pivot joints (113). The third link couples to the second link via a second of the second link's three pivot joints (114). The fourth link couples to the first link via a third of the first link's three pivot joints. The fourth link couples to the second link via a third of the first link's three pivot joints (116).

Example 8b. The exoskeleton robotic system according to any of examples 1b-7b, wherein the first direction is orthogonal to the second direction.

Example 9b. The exoskeleton robotic system according to any of examples 1b-8b comprising a sensor (110), wherein the motor and the sensor couple to the RCM mechanism via different pivot joints.

For example, see revolute joints 111, 112.

Another version of 9b. The exoskeleton robotic system according to any of examples 1b-8b comprising a sensor (110), wherein the motor and the sensor couple to the RCM mechanism via a single pivot joint.

Example 10b. The exoskeleton robotic system according to any of examples 1b-9b, wherein the RCM mechanism is a robotic shoulder joint or a robotic wrist joint.

Example 11b. The exoskeleton robotic system of example 1b, wherein: in response to actuation by the motor the RCM mechanism is configured to rotate about a center point located within a body of a user of the exoskeleton robotic system. The RCM mechanism is configured to rotate within a rotation range that is at least 40 degrees and less than 360 degrees.

Example 12b. The exoskeleton robotic system according to any of examples 1b-11b, wherein: the first link is primarily disposed in a first plane. The second link is primarily disposed in a second plane. The first plane is parallel to the second plane.

Example 1c. An exoskeleton robotic system comprising a parallelogram-based remote center of motion (RCM) joint (900). The RCM mechanism includes a first link (901) that is coupled to a second link (902), a third link (903), and a fourth link (904). Motor (907) is coupled to the third link. A first of the first, second, third, or fourth links includes a first flexure (905). The first flexure includes a first degree of freedom in a first direction (905′). A second of the first, second, third, or fourth links includes a second flexure (906), the second flexure including a second degree of freedom in a second direction (906′). The first direction is not parallel to the second direction.

See, for example, FIGS. 9A-9G.

Example 2c. The exoskeleton robotic system of example 1c, wherein the first link is a first ternary link and the second link is a second ternary link.

Example 2.1c The exoskeleton robotic system according to example 2c comprising a fifth link (917) coupled to the first link. The first flexure is included in one of the first or second links. The second flexure is included in the fifth link.

Example 2.2c. The exoskeleton robotic system according to any of examples 1c-3c, wherein in response to actuation by the motor the RCM mechanism is configured to rotate about an axis (918) located within a body of a user of the exoskeleton robotic system.

Example 3c. The exoskeleton robotic system of example 2c, wherein: the RCM mechanism includes only one degree of freedom. The only one degree of freedom is a rotational degree of freedom about the center point. The RCM mechanism does not include a second degree of freedom.

Example 4c. The exoskeleton robotic system according to any of examples 1c-3c, wherein the RCM mechanism includes at least two redundant joints.

Another version of example 4c. The exoskeleton robotic system according to any of examples 2.1c-3c, wherein the RCM mechanism includes at least two redundant joints. The at least two redundant joints are included on two of the first, second, third, fourth, or fifth links.

Example 5c. The exoskeleton robotic system according to any of examples 1c-4c, wherein the RCM mechanism is not over constrained.

Example 6c. The exoskeleton robotic system according to example 2.1c, wherein: the fifth link includes a bracket. The bracket couples to the first link via a first revolute joint (908). The bracket couples to the second link via a second revolute joint (909).

Example 7c. The exoskeleton robotic system according to example 2.1c, wherein: the fifth link includes a bracket. The bracket couples to the first link via a first revolute joint (908). The bracket couples to the second link via a second revolute joint (909). The third link couples to the first link via a third revolute joint (913). The third link couples to the second link via a fourth revolute joint (914). The fourth link couples to the first link via a fifth revolute joint (915). The fourth link couples to the second link via a sixth revolute joint (916).

Example 8c. The exoskeleton robotic system according to any of examples 1c-7c, wherein in one orientation the first direction is orthogonal to the second direction.

Another version of example 8c. The exoskeleton robotic system according to any of examples 1c-7c, wherein the first direction is not parallel to the second direction throughout the RCM mechanism's range of motion.

Example 9c. The exoskeleton robotic system according to any of examples 1c-8c comprising a sensor (910), wherein the motor and the sensor couple to the RCM mechanism via different revolute joints.

For example, see revolute joints 911, 912.

Another version of example 9c. The exoskeleton robotic system according to any of examples 1c-8c comprising a sensor, wherein the motor and the sensor couple to the RCM mechanism via a single revolute joint.

Example 10c. The exoskeleton robotic system according to any of examples 1c-9c, wherein the RCM mechanism is a robotic wrist joint.

Example 11c. The exoskeleton robotic system of example 1c, wherein: in response to actuation by the motor the RCM mechanism is configured to rotate about a center axis (918) located within a body of a user of the exoskeleton robotic system. The RCM mechanism is configured to rotate within a rotation range that is at least 40 degrees and less than 360 degrees.

Another version of example 11c. The exoskeleton robotic system of example 1c, wherein: the RCM mechanism is a parallelogram-based RCM mechanism. In response to actuation by the motor the RCM mechanism is configured to rotate about a center axis located within a body of a user of the exoskeleton robotic system. The RCM mechanism is configured to rotate through an entirety of a rotation range that includes a change point.

Example 11.1c The exoskeleton robotic system according to example 11c, wherein the first direction is never parallel to the second direction throughout the entirety of the rotation range.

Example 12c. The exoskeleton robotic system according to any of examples 1c-11c, wherein: the first link is primarily disposed in a first plane. The second link is primarily disposed in a second plane. The first plane is parallel to the second plane.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

1. An exoskeleton robotic system comprising:
a parallelogram-based remote center of motion (RCM) mechanism, the RCM mechanism including a first ternary link that is coupled to a second ternary link, a third ternary link, a fourth ternary link, a first binary link, and a second binary link;
a motor coupled to the third ternary link; and
a sensor coupled to the fourth ternary link;
wherein: (a) the first ternary link includes a first flexure the first flexure configured to act in a first direction, (b) the second ternary link includes a second flexure the second flexure configured to act in a second direction, and (c) the first direction is not parallel to the second direction.

2. The exoskeleton robotic system of claim 1, wherein in response to actuation by the motor the RCM mechanism is configured to rotate about a remote center axis located within a body of a user of the exoskeleton robotic system.

3. The exoskeleton robotic system of claim 2, wherein:
the RCM mechanism includes only one degree of freedom;
the only one degree of freedom is a rotational degree of freedom about the remote center axis;
the RCM mechanism does not include a second degree of freedom.

4. The exoskeleton robotic system according to claim 3, wherein the RCM mechanism is an RCM mechanism that includes at least two redundant joints.

5. The exoskeleton robotic system according to claim 3, wherein the RCM mechanism is not over constrained.

6. The exoskeleton robotic system according to claim 1 comprising, wherein:
the first binary link includes a bracket;
the bracket couples to the first ternary link via a first revolute joint; and
the bracket couples to the second ternary link via a second revolute joint.

7. The exoskeleton robotic system according to claim 1, wherein:
the first binary link includes a bracket;
the bracket couples to the first ternary link via a first revolute joint;
the bracket couples to the second ternary link via a second revolute joint;
the third ternary link couples to the first ternary link via a third revolute joint
the third ternary link couples to the second ternary link via a fourth revolute joint;
the fourth ternary link couples to the first ternary link via a fifth revolute joint; and
the fourth ternary link couples to the second ternary link via a sixth revolute joint

8. The exoskeleton robotic system according to claim 1, wherein the first direction is orthogonal to the second direction.

9. The exoskeleton robotic system according to claim 1, wherein:
the sensor includes a torque sensor; and
the motor and the torque sensor couple to the RCM mechanism via different revolute joints.

10. The exoskeleton robotic system according to claim 1, wherein the RCM mechanism is a robotic shoulder joint or a robotic wrist joint.

11. The exoskeleton robotic system of claim 1, wherein:
the RCM mechanism is a parallelogram-based RCM mechanism;
in response to actuation by the motor the RCM mechanism is configured to rotate about a center axis located within a body of a user of the exoskeleton robotic system;
the RCM mechanism is configured to rotate within a rotation range that is at least 40 degrees and less than 180 degrees.

12. The exoskeleton robotic system according to claim 11, wherein the first direction is never parallel to the second direction throughout the entirety of the rotation range.

13. The exoskeleton robotic system according to claim 1, wherein:
the first ternary link is primarily disposed in a first plane;
the second ternary link is primarily disposed in a second plane;
the first plane is parallel to the second plane.

14. An exoskeleton robotic system comprising:
a parallelogram-based remote center of motion (RCM) joint (200), the RCM joint including a first link that is coupled to a second link, a third link, and a fourth link;
a motor coupled to the third link;
wherein: (a) a first of the first, second, third, or fourth links includes a first flexure, the first flexure including a first degree of freedom in a first direction, (b) a second of the first, second, third, or fourth links includes a second flexure the second flexure including a second degree of freedom in a second direction and (c) the first direction is not parallel to the second direction.

15. The exoskeleton robotic system of claim 14, wherein the first link is a first ternary link and the second link is a second ternary link.

16. The exoskeleton robotic system according to claim 15, wherein:
the first flexure is included in one of the first or second links; and
the second flexure is included in one of the third or fourth links.

17. The exoskeleton robotic system according to claim 14, wherein in response to actuation by the motor the RCM joint is configured to rotate about a center point located within a body of a user of the exoskeleton robotic system.

18. The exoskeleton robotic system of claim 17, wherein:
the RCM joint includes only one degree of freedom;
the only one degree of freedom is a rotational degree of freedom about the center point;
the RCM joint does not include a second degree of freedom.

19-25. (canceled)

26. The exoskeleton robotic system of claim 14, wherein:
in response to actuation by the motor the RCM joint is configured to rotate about a center axis located within a body of a user of the exoskeleton robotic system;
the RCM joint is configured to rotate within a rotation range that is at least 40 degrees and less than 360 degrees.

27. The exoskeleton robotic system according to claim 26, wherein the first direction is never parallel to the second direction throughout the entirety of the rotation range.

28-40. (canceled)
